(19) United States
(12) Patent Application Publication
(10) Pub. No.: US 2006/0217590 A1
(43) Pub. Date: Sep. 28, 2006

(54) Artificial insemination device for domestic animals

(76) Inventor: Dong-Soo Tack, Gyeonggi-do (KR)

Correspondence Address: Jeffrey J. King;GRAYBEAL JACKSON HALEY LLP, Suite 350, 155 - 108th Avenue NE, Bellevue, WA, 98004-5973, US

(21) Appl. No.: 11/089,813

(22) Filed: Mar. 25, 2005

(51) Int. Cl. A61B 17/43; A61D 7/00; A01K 29/00

(52) U.S. Cl. 600/35; 119/174

(57) ABSTRACT

An artificial insemination device for domestic animals comprises: a body having a handle and provided with an insert hole at one side thereof; an insert tube detachably coupled to the body and formed as a hollow cylindrical shape; a viewing unit mounted in the body; a semen inject tube inserted into the insert hole and the insert tube and having a length longer than the insert tube, for injecting semen; and a housing unit provided in the body for preventing an animal's secretion introduced through the insert tube from being introduced into a battery mounted at the handle. Accordingly, internal units of the device are protected from an animal's secretion, the device is sanitarily used by easily exchanging components, and usage convenience is increased.

### BACKGROUND OF THE INVENTION

1. Field of the Invention

The present invention relates to an artificial insemination device for domestic animals, and more particularly, to an artificial insemination device for domestic animals capable of protecting internal units thereof from an animal's secretion, capable of being sanitarily used by easily exchanging components, and capable of increasing usage convenience.

2. Description of the Conventional Art

As the conventional method for an artificial insemination for domestic animals such as cattle, a horse, a deer, etc. an injection tube formed as a syringe type was being widely used. At the time of using the injection tube, a semen straw is inserted into a cylindrical injection tube to which a piston is coupled and then is inserted into an animal's cervix entrance. Then, the piston is pushed thereby to inject the semen inside the semen straw into the cervix.

However, the syringe type injection tube has the following problems. First, since the cervix entrance can not be certified with a user's naked eyes, the user had to sensually insert the syringe type injection tube into the cervix by using his hand. In this case, even a skilled person can not insert the injection tube into a precise position of the cervix, an operation speed is slow, the domestic animal is damaged, etc.

In order to solve the problems, one invention for the user to insert an injection tube into a domestic animal's vagina with certifying the inner state of the vagina with his naked eyes by using a periscope was disclosed in U.S Pat. No. 6,059,716. FIG. 1 is a sectional view showing the conventional artificial insemination device for domestic animals disclosed in U.S Pat. No. 6,059,716, and FIG. 2 is a sectional view taken along line II-II in FIG. 1.

As shown, the conventional artificial insemination device for domestic animals includes an in-vagina insert unit 10 having a guide, passage 11 therein, and a semen injection unit 20 for inserting semen into a domestic animal's body via the guide passage 11.

The in-vagina insert unit 10 includes an insert body 15 having an upward extension 16 extendingly formed to mount a first mirror 18a and a second mirror 18b facing each other therein, and an insert tube 12 formed at the end of the insert body 15.

The insert body 15 is provided with an insert hole 35 for inserting the semen injection unit 20 at a rear side thereof, and the insert hole 35 is opened/closed by a stopper pin 40. The stopper pin 40 is provided with an inclined surface 41 at the front end thereof. A coil spring 42 is formed at the rear side of the stopper pin 40, so that the stopper pin 40 is supported to receive a force that is always towards the upper side. Under the construction, when the semen injection unit 20 is not inserted into the insert hole 35, the stopper pin 40 is in contact with the first mirror 18a, and when the semen injection unit 20 is inserted into the insert hole 35, the coil spring 42 is compressed thereby to open the insert hole 35.

The insert tube 12 is provided with a lamp 13 at the front side thereof. The lamp 13 is connected to a battery 48 and is turned on/off by a switch 45.

The insert tube 12 is constructed to have various lengths protruded from the insert body 15 according to a depth of an animal's vagina.

More concretely, first, second, and third locking holes 17a, 17b, and 17c are formed with a certain interval at an upper end of the insert body 15, and an engagement hole 12a is formed at a rear side of the insert tube 12. Then, one of the first, second, and third locking holes is made to correspond to the engagement hole 12a so that the insert tube 12 can be protruded as a desired length. Then, a locking member 19 is inserted into the engagement hole 12a thereby to be fixed.

As shown in FIG. 2, the locking member 19 includes a support portion 19a having a lower portion opened as a ring shape and inserted into the insert tube 12, and a protrusion portion 19b upwardly extending from the upper side of the support portion 19a and inserted into the locking holes 17a, 17b, and 17c and the engagement hole 12a.

The semen insert unit 20 is provided with a guide/nozzle member 22 formed as a conical shape at the front end thereof. A semen injection tube (not shown) is inserted into the semen injection unit 20 to be inserted into an animal's vagina.

An operation of the conventional artificial insemination device for domestic animals will be explained as follows.

First, one of the first, second, and third locking holes 17a, 17b, and 17c is selected so that the insert tube 12 can be protruded as a proper length according to a depth of an animal's vagina, and then is made to be identical to the engagement hole 12a. Then, the locking member 19 is inserted into the engagement hole 12a. In this case, the locking member 19 is in a state of being supported in the insert tube 12 by the support portion 19a.

Then, the switch 45 is turned on to turn on the lamp 13. Under this state, the insert tube 12 and the insert body 15 are inserted into the animal's vagina. Since the inside of the animal's vagina is reflected on the second mirror 18b via the first mirror 18a, the operator can operate with observing a position of the cervix in which semen is inseminated. After certifying a position of the cervix in which semen is inseminated, the operator reaches the semen inject unit 20 into the cervix via the guide passage 11 of the in-vagina insert unit 10. Then, semen is injected into the cervix by the semen injection tube (not shown) inside the semen inject unit 20. The semen is sprayed through the guide/nozzle member 22 thereby to be inseminated.

The conventional artificial insemination device for domestic animals has an advantage that the user can operate with certifying the inner state of the animal's vagina with his naked eyes by using a principle of a periscope. However, the conventional artificial insemination device for domestic animals has the following problems.

First, since a force is concentrated on the locking member 19 while the insert tube 12 and the insert body 15 are inserted into the animal's vagina, the protrusion portion 19b is frequently damaged. That is, since the animal's vagina is contracted at an estrous time, a strong force is required at the time of inserting the insert tube 12 and the insert body 15 into the animal's vagina. The strong force is concentrated on the protrusion portion 19b of the locking member 19 for coupling the insert tube 12 and the insert body 15 each other. Therefore, said problem is caused.

Also, the animal's vagina secretion backflows via the guide passage 11 thereby to reach the battery 48 through a gap between the stopper pin 40 and the semen inject unit 20. The secretion causes the battery 48 to be malfunctioned and thereby the battery 48 has to be frequently exchanged.

Additionally, since the insert tube 12 and the insert body 15 become dirty after a single usage, the user has a difficulty in cleaning them. Especially, it is difficult to clean the inside of the insert body 15 and parts where the stopper pin 40 and the battery 48 are mounted. Also, when the insert tube 12 and the insert body 15 are re-used, the animal's virus infection may be caused due to dirtiness of the insert tube 12 and the insert body 15.

### SUMMARY OF THE INVENTION

Therefore, an object of the present invention is to provide an artificial insemination device for domestic animals capable of protecting internal units thereof from secretion of domestic animals, capable of being sanitarily used by easily exchanging components, and capable of increasing usage convenience.

To achieve these and other advantages and in accordance with the purpose of the present invention, as embodied and broadly described herein, there is provided an artificial insemination device for domestic animals comprising: a body having a handle and provided with an insert hole at one side thereof; an insert tube detachably coupled to the body and formed as a hollow cylindrical shape; a viewing unit mounted in the body; a semen inject tube inserted into the insert hole and the insert tube and having a length longer than the insert tube, for injecting semen; and a housing unit provided in the body for preventing an animal's secretion introduced through the insert tube from being introduced into a battery mounted at the handle.

The foregoing and other objects, features, aspects and advantages of the present invention will become more apparent from the following detailed description of the present invention when taken in conjunction with the accompanying drawings.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 is a sectional view showing an artificial insemination device for domestic animals disclosed in U.S Pat. No. 6,059,716 in accordance with the conventional art;

FIG. 2 is a sectional view taken along line II-II in FIG. 1;

FIG. 3 is a perspective view showing an artificial insemination device for domestic animals according to the present invention;

FIG. 4 is a sectional view showing the artificial insemination device for domestic animals according to the present invention;

FIG. 5 is a lateral view showing the artificial insemination device for domestic animals according to the present invention;

FIG. 6 is a frontal view showing the artificial insemination device for domestic animals under a state a handle is opened according to the present invention;

FIG. 7 is an enlarged section view showing a housing unit according to a second embodiment of the present invention;

FIG. 8 is an enlarged section view showing a housing unit according to a third embodiment of the present invention; and

FIG. 9 is an enlarged section view showing a housing unit according to a fourth embodiment of the present invention.

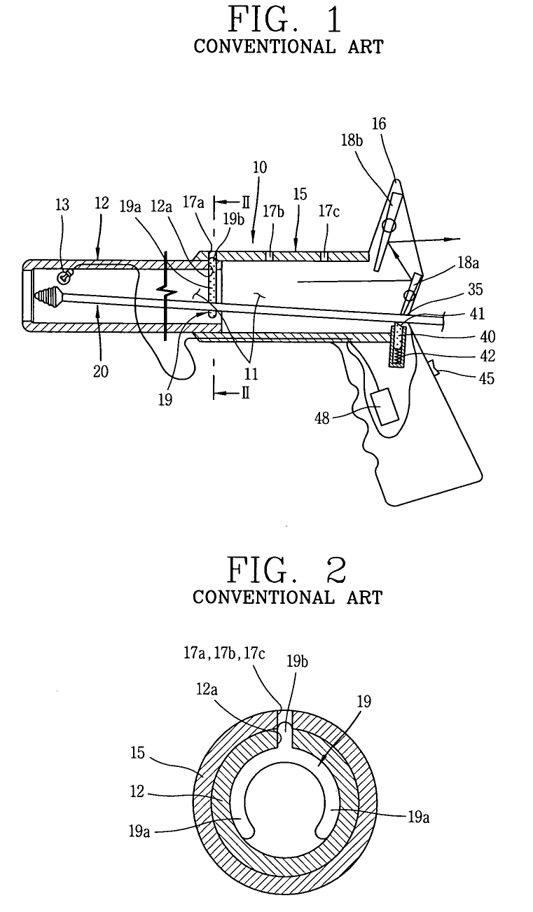

### DETAILED DESCRIPTION OF THE PREFERRED EMBODIMENTS

Reference will now be made in detail to the preferred embodiments of the present invention, examples of which are illustrated in the accompanying drawings.

Even if a plurality of preferred embodiments may exist in the present invention, the most preferred embodiment will be explained hereinafter.

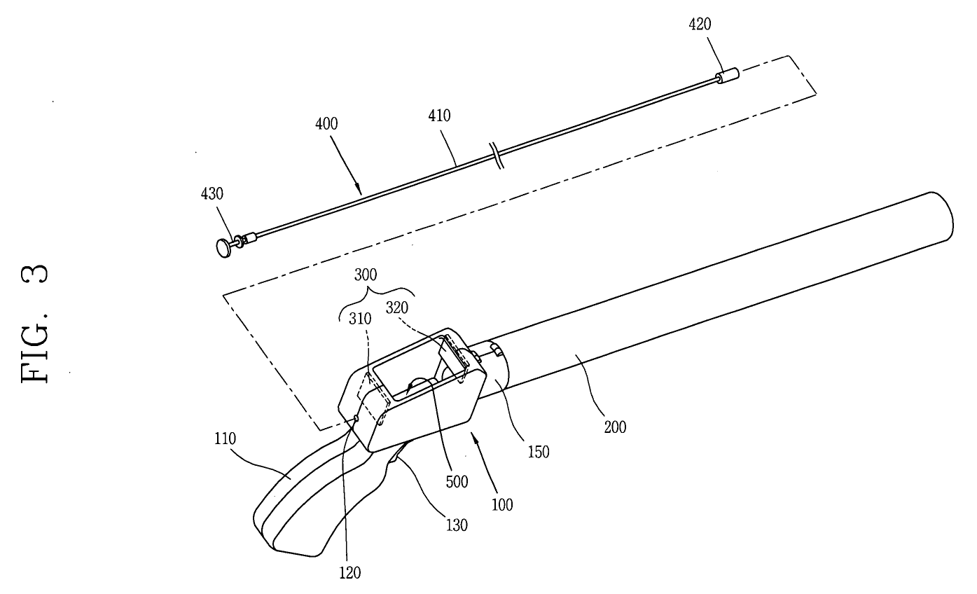

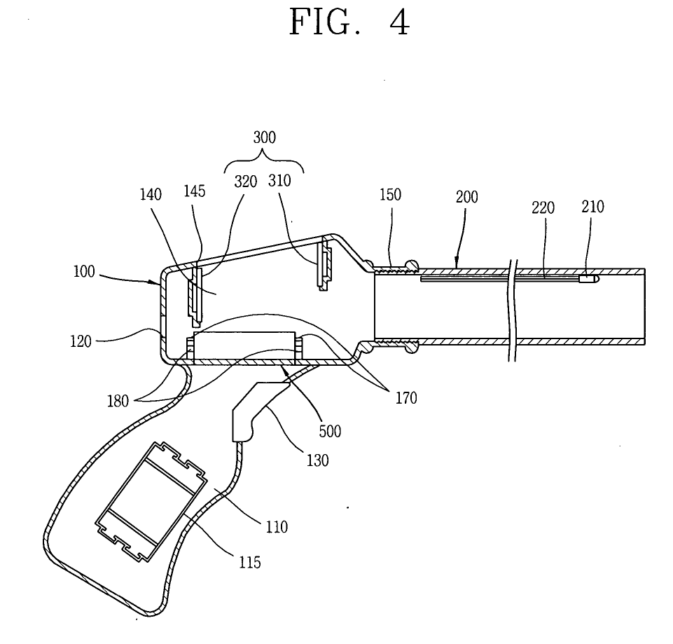

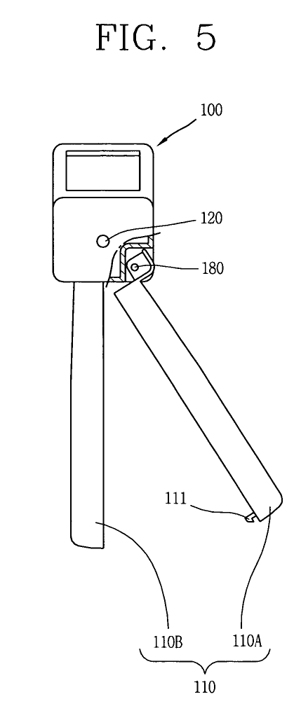

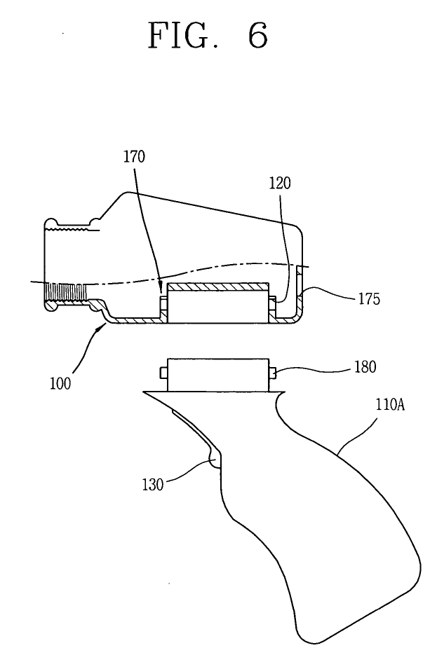

FIG. 3 is a perspective view showing an artificial insemination device for domestic animals according to the present invention, FIG. 4 is a sectional view showing the artificial insemination device for domestic animals according to the present invention, FIG. 5 is a lateral view showing the artificial insemination device for domestic animals according to the present invention, and FIG. 6 is a frontal view showing the artificial insemination device for domestic animals under a state a handle is opened according to the present invention.

As shown, the artificial insemination device for domestic animals according to the present invention comprises: a body 100 having a handle 110 and provided with an insert hole 120 at one side thereof; an insert tube 200 detachably coupled to the body 100 and formed as a hollow cylindrical shape; a viewing unit 300 mounted in the body 100; a semen inject tube 400 inserted into the insert hole 120 and the insert tube 200 and having a length longer than the insert tube 200, for injecting semen; and a housing unit 500 provided in the body 100 for preventing an animal's secretion introduced through the insert tube 200 from being introduced into the battery mounted at the handle.

The body 100 includes a viewing unit mounting portion 140 having a space for mounting the viewing unit 300, the handle 110 integrally formed at a lower side of the viewing unit mounting portion 140, and a connection portion 150 detachably connected to the insert tube 200.

The viewing unit mounting portion 140 is provided with an open portion 145 of which upper side is opened, so that the user can observe the viewing unit 300 with his naked eyes.

A switch 130 for operating a lamp 210 that will be later explained is coupled to outside of the handle 110. A battery chamber 115 for mounting the battery is formed in the handle 110. A battery is coupled to the battery chamber 115.

As shown in FIGS. 5 and 6, the handle 110 includes a rotary portion 110A coupled to the body 100 to be rotatable, and a fixed portion 110B coupled to the rotary portion 110A and integrally formed at the body 100. The rotary portion 110A is rotatably coupled to the end of a support axis 170 extending from an inner surface of the body 100. More concretely, a protrusion portion 180 protruded at both sides of an upper end of the rotary portion 110A is rotatably inserted into a hole 175 formed at the end of the support axis 170. Also, a hook 111 is formed at a lower end of the rotary portion 110A. The hook 111 is locked by a stopping groove (not shown) formed at a lower end of the fixed portion 110B.

The hook 111 is separated from the stopping groove to be rotated centering around the protrusion portion 180, and the battery chamber 115 is exposed to outside, thereby exchanging the battery.

The connection portion 150 detachably forms a screw thread thereby to perform a pipe connection. That is, a screw thread is provided at a rear end of the insert tube 200, and a screw thread corresponding to the screw thread of the insert tube 200 is formed at the connection portion 150 thereby to construct the connection portion 150 to be detachable.

It is also possible to detachably construct the connection portion 150 by a bolt. The connection portion 150 can be detachable by a hook. The connection portion 150 can be detachably constructed by various methods.

The connection portion 150 includes a contact point to be electrically connected to the lamp 210 and the battery mounted in the battery chamber 115.

The contact point is preferably formed as a plate shape.

The insert tube 200 is preferably formed of foam plastic that is harmless to the animal and cheap enough to be discarded after a single usage.

The lamp 210 for lighting inside of the animal is coupled to the inner front side of the insert tube 200. A wire 220 for connecting the lamp 210 and the battery is attached to an inner wall of the insert tube 200. The wire 220 can be buried at the inner wall of the insert tube 200.

The viewing unit 300 includes a first mirror 310 formed at a rear end of the viewing unit mounting portion 140 with a certain angle for reflecting the animal's inserted portion, and a second mirror 320 formed at a front end of the viewing unit mounting portion 140 and facing the first mirror 310. The inner state of the animal's inserted portion is reflected on the first mirror 310 and the second mirror 320, so that the user can operate with observing the inside of the animal's vagina through the first and second mirrors 310 and 320.

The viewing unit 300 can be variously constructed. For example, a camera can be installed at a position where the lamp 210 is installed, and a monitor can be installed outside the body 100 thereby to observe the inside of the animal's vagina.

The first mirror 310 and the second mirror 320 are respectively provided with a heat wire at a rear surface thereof to heat them, thereby preventing a dew condensation phenomenon. That is, when an external temperature falls below zero in winter, humid heat inside the animal's vagina corresponding to 35° C.˜40° C. is transmitted to the first mirror 310 and the second mirror 320 through the insert tube 200. Accordingly, a dew condensation is generated on the first mirror 310 and the second mirror 320 to blur the mirrors, so that the user has to wipe the dew condensation or has to use a dew condensation preventing material. The heat wire uses the battery mounted in the battery chamber 115 as power. Preferably, the switch 130 is implemented as a slide method, etc., thereby to turn on/off the lamp 210 and the heat wire simultaneously or selectively.

The semen inject tube 400 includes an inject tube 410 having a length longer than the insert tube 200 and containing an animal's semen therein, a semen spray unit 420 formed at a front side of the inject tube 410 and having a nozzle therein to spray the animal's semen, and a plunger 430 formed at a rear side of the inject tube 410 for pressurizing the animal's semen contained in the inject tube 410.

Since the inject tube 410 has a length longer than the insert tube 200, the inject tube 410 is inserted into the animal's cervix in a state of being protruded from the insert tube 200.

The inject tube 410 may further include an additional tube for containing the animal's semen therein. In the case, the plunger 430 is connected to the tube so that the semen can flow in the longitudinal direction of the tube at the time of pressurizing the semen by the plunger 430.

The front end of the semen spray unit 420 is rounded for a smooth insertion of semen.

The housing unit 500 is formed between the viewing unit mounting portion 140 and the handle 110 having the battery chamber 115. The housing unit 500 serves as a partition between the viewing unit mounting portion 140 and the handle 110, thereby preventing secretion introduced through the insert tube 200 from being introduced into the battery chamber 115. Accordingly, the secretion exists only in the viewing unit mounting portion 140, thereby easily cleaning the viewing unit mounting portion 140 after separating the insert tube 200 therefrom.

The housing unit 500 is preferably formed integrally with the body 100 and the handle 110 by using a molding.

An operation and effects of the present invention will be explained as follows.

when an animal to be artificially inseminated reaches an estrous time, the insert tube 200 is inserted into the animal's vagina and then the switch 130 is adjusted to turn on the lamp 210. With observing the inside of the animal's vagina by the first mirror 310 and the second mirror 320, the user inserts the semen inject tube 400 in which the animal's semen is contained into the insert hole 120 and the insert tube 200 thereby to find a precise insemination position and then injects the animal's semen. At this time, the animal's secretion backflows through the insert tube 200. However, the animal's secretion does not reach the battery chamber 115 in which the battery is mounted by the housing unit 500 but stays in the viewing unit mounting portion 140 of the body 100. Accordingly, the problem that the battery has to be frequently exchanged due to the secretion can be solved.

Also, in the present invention, the connection portion 150 for coupling the body 100 and the insert tube 200 is detachably constructed without a locking member, thereby preventing the conventional problem that force is concentrated on the locking member to damage the locking member.

Additionally, in the present invention, the body 100 and the insert tube 200 are detachable constructed, and the insert tube 200 that is cheap and harmless to the animal is discarded after a single usage. Accordingly, the animal's virus injection due to re-use of the insert tube 200 can be prevented, and a cleaning operation can be facilitated.

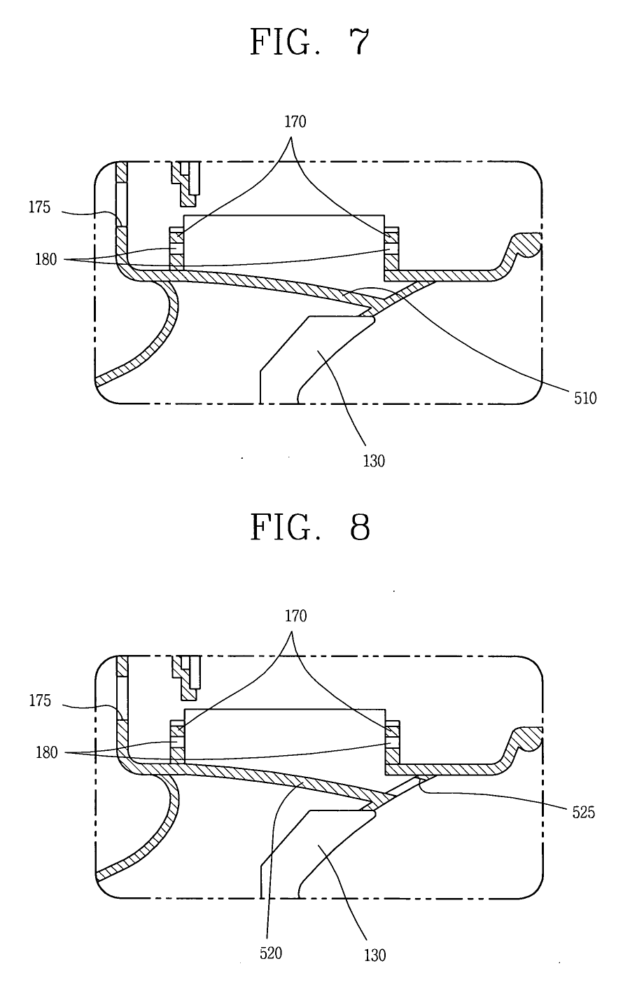

Another embodiment of the housing unit is illustrated in FIG. 7. FIG. 7 is an enlarged section view showing the housing unit according to a second embodiment of the present invention.

As shown, the housing unit 510 is formed to be inclined forward with a certain angle. The housing unit stores the animal's secretion, and the secretion is collected into a predetermined space of the housing unit 510 along a wall surface of the housing unit 510 thereby to facilitate a cleaning operation.

FIG. 8 is an enlarged section view showing the housing unit according to a third embodiment of the present invention.

As shown, the housing unit 520 is formed to be inclined forward with a certain angle. The housing unit is provided with a through hole 525 penetrating the handle at a position facing the inclined surface thereof. The animal's secretion is collected into a predetermined space of the housing unit 520 along a wall surface of the housing unit 520, and then is discharged out via the through hole 525.

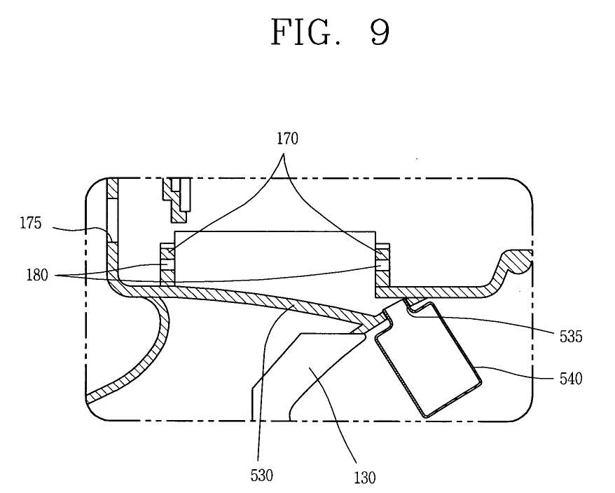

FIG. 9 is an enlarged section view showing the housing unit according to a fourth embodiment of the present invention.

As shown, the housing unit 530 is formed to be inclined forward with a certain angle. The housing unit is provided with a through hole 535 penetrating the handle at a position facing the inclined surface thereof. A storing container 540 is coupled to the through hole 535. The animal's secretion is collected into a predetermined space of the housing unit 530 along a wall surface of the housing unit 530, and then is collected into the storing container 540 via the through hole 535. Accordingly, the problem that the user's hands or clothes become dirty while the animal's secretion is discharged out can be solved.

As the present invention may be embodied in several forms without departing from the spirit or essential characteristics thereof, it should also be understood that the above-described embodiments are not limited by any of the details of the foregoing description, unless otherwise specified, but rather should be construed broadly within its spirit and scope as defined in the appended claims, and therefore all changes and modifications that fall within the metes and bounds of the claims, or equivalence of such metes and bounds are therefore intended to be embraced by the appended claims.

What is claimed is:

1. An artificial insemination device for domestic animals comprising:
a body having a handle and provided with an insert hole at one side thereof;
an insert tube detachably coupled to the body and formed as a hollow cylindrical shape;
a viewing unit mounted in the body;
a semen inject tube inserted into the insert hole and the insert tube and having a length longer than the insert tube, for injecting semen; and
a housing unit provided in the body for preventing an animal's secretion introduced through the insert tube from being introduced into a battery mounted at the handle.

2. The device of claim 1, wherein the body comprises:
a viewing unit mounting portion having a space for mounting the viewing unit;
a handle integrally formed at a lower side of the viewing unit mounting portion; and
a connection portion detachably connected to the insert tube.

3. The device of claim 2, wherein the viewing unit mounting portion is provided with an open portion of which upper side is opened so that the user can observe the viewing unit with his naked eyes.

4. The device of claim 1, wherein the handle comprises:
a rotary portion coupled to the body to be rotatable; and
a fixed portion coupled to the rotary portion and integrally formed at the body.

5. The device of claim 2, wherein the connection portion forms a screw thread therein thereby to be detachable from the insert tube having a screw thread corresponding to the screw thread of the connection portion.

6. The device of claim 2, wherein the connection portion is detachably constructed by a bolt.

7. The device of claim 2, wherein the connection portion is detachably constructed by a hook.

8. The device of claim 2, wherein the viewing unit comprises:
a first mirror formed at a rear end of the viewing unit mounting portion with a certain angle for reflecting an animal's inserted portion; and
a second mirror formed at a front end of the viewing unit mounting portion and facing the first mirror.

9. The device of claim 8, wherein the first mirror and the second mirror are respectively provided with a heat wire at a rear surface thereof to heat them.

10. The device of claim 1, wherein the semen inject tube comprises:
an inject tube having a length longer than the insert tube and containing an animal's semen therein;
a semen spray unit formed at a front side of the inject tube and having a nozzle therein to spray the animal's semen; and
a plunger formed at a rear side of the inject tube for pressurizing the animal's semen contained in the inject tube.

11. The device of claim 10, wherein the inject tube further comprises a tube for containing the animal's semen therein.

12. The device of claim 2, wherein the housing unit is formed between the viewing unit mounting portion and the handle to serve as a partition therebetween.

13. The device of claim 1, wherein the housing unit is inclined with a certain angle.

14. The device of claim 1, wherein the housing unit is inclined with a certain angle, and is provided with a through hole penetrating the handle at a position facing the inclined surface thereof.

15. The device of claim 1, wherein the housing unit is inclined with a certain angle, is provided with a through hole penetrating the handle at a position facing the inclined surface thereof, and a storing container is coupled to the through hole.
